(19) United States
(12) Patent Application Publication
(10) Pub. No.: US 2024/0127964 A1
(43) Pub. Date: Apr. 18, 2024

(54) DATA PROCESSING DEVICE, SYSTEM, DATA PROCESSING METHOD, AND RECORDING MEDIUM

(71) Applicant: NEC Corporation, Tokyo (JP)

(72) Inventors: Kenichiro Fukushi, Tokyo (JP); Chenhui Huang, Tokyo (JP); Zhenwei Wang, Tokyo (JP); Fumiyuki Nihey, Tokyo (JP); Kentaro Nakahara, Tokyo (JP)

(73) Assignee: NEC Corporation, Tokyo (JP)

(21) Appl. No.: 18/543,057

(22) Filed: Dec. 18, 2023

Related U.S. Application Data
(63) Continuation of application No. 17/918,151, filed on Oct. 11, 2022.

(51) Int. Cl. G16H 50/70; G16H 50/30

(52) U.S. Cl. CPC G16H 50/70; G16H 50/30

(57) ABSTRACT

A data processing device that includes a classification unit that classifies at least one piece of living-body information data into at least one group based on an attribute of at least one user, the at least one piece of living-body information data including a sensor value relating to a living body of the user, and a measurement time and a measurement position of the sensor value, and a learning unit that generates, for each of the group, a model for estimating interpolation data for interpolating a deficit of the living-body information data using a correlation among the sensor value included in the living-body information data, the measurement time, and the measurement position.

This application is a Continuation of U.S. application Ser. No. 17/918,151 filed on Oct. 11, 2022, which is a National Stage Entry of PCT/JP2020/016916 filed on Apr. 17, 2020, the contents of all of which are incorporated herein by reference, in their entirety.

### TECHNICAL FIELD

The present invention relates to a data processing device and the like that process data relating to a living body.

### BACKGROUND ART

In daily life, a technology for collecting data on a living body is required. Such data is measured by a sensor or the like mounted on a wearable device or the like worn by a user. In a case where a wearable device is used, the frequency of data measurement may decrease due to restriction of battery capacity, influence of body motion noise, a user's lifestyle, forgetting to wear, or the like. When the measurement frequency of data decreases, there is a possibility that data necessary for monitoring the living-body information is lost.

PTL 1 discloses a technique for timely measuring/collecting a living-body index necessary for evaluating a health condition. In the method of PTL 1, when the measurement data of the living-body index cannot be acquired, a user is requested to re-measure the living-body index. In the method of PTL 1, in a case where the deficit in the measurement data occurs, the deficit portion of the data set including the deficit is interpolated using the accumulated past data set. For example, in the method of PTL 1, a data set to be used for interpolation is selected by focusing on the similarity of the data set.

### CITATION LIST

### Patent Literature

[PTL 1] JP 2010-142273 A

### SUMMARY OF INVENTION

### Technical Problem

In the method of PTL 1, even in a case where a deficit occurs in the measurement data, a deficit portion can be interpolated. In the method of PTL 1, when interpolating a data missed portion using a past data set, conditions such as time and place when the data set was acquired are not included. Therefore, in the method of PTL 1, when conditions such as the time and place at which the past data set used for interpolation was acquired are different, the living-body index may be greatly different. Therefore, in the method of PTL 1, the precision of interpolated data sometimes decreases.

An object of the present invention is to provide a data processing device and the like capable of interpolating a deficit in data relating to a living body with high precision.

### Solution to Problem

A data processing device according to an aspect of the present invention includes: a classification unit configured to classify at least one piece of living-body information data into at least one group based on an attribute of at least one user, the at least one piece of living-body information data including a sensor value relating to a living body of the user, and a measurement time and a measurement position of the sensor value; and a learning unit configured to generate, for each of the group, a model for estimating interpolation data for interpolating a deficit of the living-body information data using a correlation among the sensor value included in the living-body information data, the measurement time, and the measurement position.

A data processing method according to an aspect of the present invention includes: classifying at least one piece of living-body information data into at least one group based on an attribute of at least one user, the at least one piece of living-body information data including a sensor value relating to a living body of the user, and a measurement time and a measurement position of the sensor value; and generating, for each of the group, a model for estimating interpolation data for interpolating a deficit of the living-body information data using a correlation among the sensor value included in the living-body information data, the measurement time, and the measurement position.

A program according to an aspect of the present invention causes a computer to execute: classifying at least one piece of living-body information data into at least one group based on an attribute of at least one user, the at least one piece of living-body information data including a sensor value relating to a living body of the user, and a measurement time and a measurement position of the sensor value; and generating, for each of the group, a model for estimating interpolation data for interpolating a deficit of the living-body information data using a correlation among the sensor value included in the living-body information data, the measurement time, and the measurement position.

### Advantageous Effects of Invention

According to the present invention, it is possible to provide a data processing device and the like capable of interpolating a deficit in data relating to a living body with high precision.

### BRIEF DESCRIPTION OF DRAWINGS

FIG. 1 is a block diagram illustrating an example of a configuration of a data processing device according to a first example embodiment.

FIG. 2 is an example of an attribute table stored in a storage unit of the data processing device according to the first example embodiment.

FIG. 3 is another example of the attribute table stored in the storage unit of the data processing device according to the first example embodiment.

FIG. 4 is an example of a living-body information table generated by an aggregation unit of the data processing device according to the first example embodiment.

FIG. 5 is a conceptual diagram for explaining generation of a model by the data processing device according to the first example embodiment.

FIG. 6 is a conceptual diagram for explaining estimation of living-body information data by the data processing device according to the first example embodiment.

FIG. 7 is a flowchart regarding generation of a model by the data processing device according to the first example embodiment.

FIG. 8 is a flowchart regarding estimation of living-body information data by the data processing device according to the first example embodiment.

FIG. 9 is another example of the living-body information table generated by the aggregation unit of the data processing device according to the first example embodiment.

FIG. 10 is still another example of the living-body information table generated by the aggregation unit of the data processing device according to the first example embodiment.

FIG. 11 is a block diagram illustrating an example of a configuration of a system of a second example embodiment.

FIG. 12 is a conceptual diagram for explaining an arrangement of a wearable device included in a system of the second example embodiment.

FIG. 13 is a block diagram illustrating an example of a configuration of the wearable device included in the system of the second example embodiment.

FIG. 14 is a block diagram illustrating an example of a configuration of a terminal device included in the system of the second example embodiment.

FIG. 15 is a block diagram illustrating an example of a configuration of a data processing device included in the system of the second example embodiment.

FIG. 16 is a flowchart for explaining an initial setting phase in the system of the second example embodiment.

FIG. 17 is a flowchart for explaining a measurement phase in the system of the second example embodiment.

FIG. 18 is a flowchart for explaining a data processing phase in the system of the second example embodiment.

FIG. 19 is a block diagram illustrating an example of a configuration of a system of a third example embodiment.

FIG. 20 is an example of a living-body information table generated by the data processing device according to the third example embodiment.

FIG. 21 is a block diagram illustrating an example of a configuration of a data processing device according to a fourth example embodiment.

FIG. 22 is a conceptual diagram for explaining generation of a model and estimation of an evaluation value by the data processing device according to the fourth example embodiment.

FIG. 23 is a conceptual diagram for explaining an example of displaying content based on an evaluation value estimated by the data processing device according to the fourth example embodiment on a screen of a terminal device.

FIG. 24 is a conceptual diagram for explaining another example of displaying content based on an evaluation value estimated by the data processing device according to the fourth example embodiment on a screen of a terminal device.

FIG. 25 is a conceptual diagram for explaining still another example of displaying content based on an evaluation value estimated by the data processing device according to the fourth example embodiment on a screen of a terminal device.

FIG. 26 is a block diagram illustrating an example of a configuration of a model generation device according to a fifth example embodiment.

FIG. 27 is a block diagram illustrating an example of a configuration of an estimation device according to a sixth example embodiment.

FIG. 28 is a block diagram illustrating an example of hardware for achieving the data processing device according to each embodiment.

### EXAMPLE EMBODIMENT

Hereinafter, embodiments of the present invention will be described with reference to the drawings. However, the embodiments described below have technically preferable limitations for carrying out the present invention, but the scope of the invention is not limited to the following. In all the drawings used in the following description of the embodiment, the same reference numerals are given to the same parts unless there is a particular reason. In the following embodiments, repeated description of similar configurations and operations may be omitted. The directions of the arrows in the drawings illustrate an example, and do not limit the directions of data, signals, and the like between blocks.

### First Example Embodiment

First, a configuration of a data processing device according to a first example embodiment of the present invention will be described with reference to the drawings. The data processing device of the present example embodiment generates a model for estimating interpolation data that interpolates the deficit in living-body information data including a value of sensor data (also referred to as a sensor value) measured by a wearable device or the like worn by a user. The living-body information data is data in which at least a sensor value, an identifier for identifying a user (also referred to as a user identifier), a measurement time and a measurement position of the sensor value are associated. For example, the data processing device according to the present example embodiment generates a model by machine learning.

### Configuration

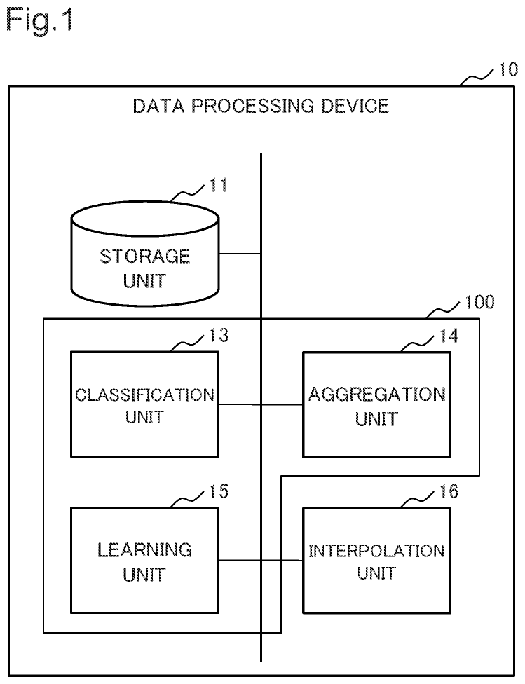

FIG. 1 is a block diagram illustrating an example of a configuration of a data processing device 10 according to the present example embodiment. The data processing device 10 includes a storage unit 11, a classification unit 13, an aggregation unit 14, a learning unit 15, and an interpolation unit 16. The classification unit 13, the aggregation unit 14, and the learning unit 15 constitute a model generation device 100.

The storage unit 11 stores attribute data of each of a plurality of users and living-body information data of the plurality of users. The attribute data and the living-body information data are stored in advance in the storage unit 11. The attribute data is acquired from a terminal device or the like (not illustrated) communicably connected via a network such as the Internet or an intranet in an initial setting phase. The living-body information data is acquired from a terminal device or the like (not illustrated) communicably connected to the wearable device or the like in the measurement phase.

The living-body information data includes a sensor value measured by a wearable device or the like worn by the user, a user identifier, a measurement time of the sensor value, and a measurement position. The user identifier is given by either the wearable device or the terminal device. The measurement time and the measurement position may be given when the wearable device or the like measures the sensor data, or may be given by a terminal device that acquires the sensor data measured by the wearable device or the like. For example, in a case where the terminal device is a mobile terminal such as a smartphone, a tablet, or a mobile phone, the measurement position of the sensor value can be acquired by using a position measurement function (for example, GPS: Global Positioning System) of the mobile terminal.

The attribute data is data in which a user identifier given to each user is associated with an attribute of each user. For example, the attribute of the user includes data such as gender, height, and weight of the user. The attribute data stored in the storage unit 11 may be updated at a certain timing according to the update by the user via the terminal device.

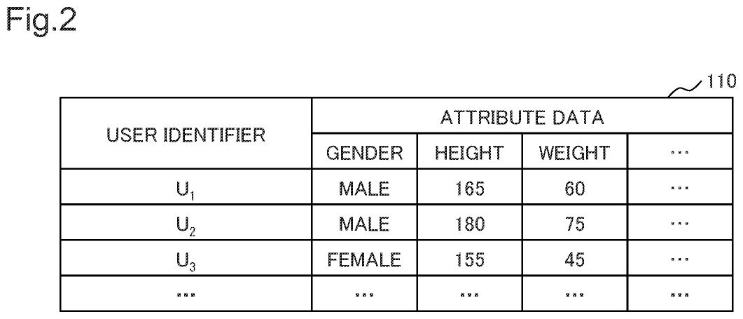

FIG. 2 is an example of a table (attribute table 110) summarizing attribute data. As in the attribute table 110, the attribute data is stored in association with the user identifier (U₁, U₂, U₃, . . . ) of each of the plurality of users.

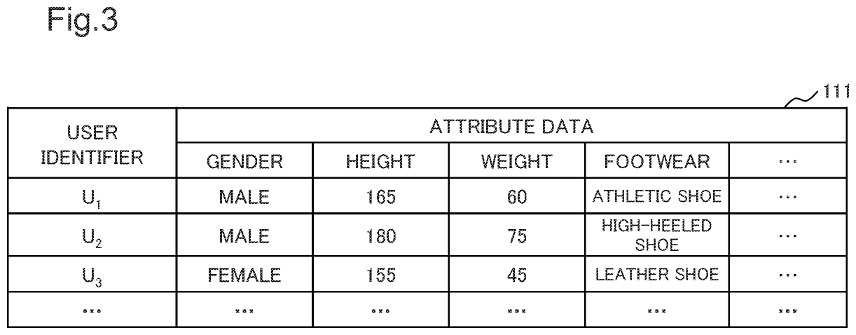

FIG. 3 is an example of a table (attribute table 111) summarizing attribute data to which the type of footwear worn by the user (athletic shoes, high-heeled shoes, leather shoes, and the like) is added. In the attribute table 111, the type of footwear worn by the user is added in addition to the living-body attribute of the user associated with the user identifier (U₁, U₂, U₃, . . . ) of each of the plurality of users. An attribute other than the type of footwear may be added to the attribute data as long as the precision of the deficit data interpolated in the living-body information data is improved. For example, an attribute of an object (for example, clothes, hats, gloves, masks, and the like) on which the wearable device is installed may be added to the attribute data. For example, users having similar attributes and wearing the same type of footwear are highly likely to measure similar sensor values. Therefore, if the living-body information data is classified including the type of the footwear or the model is generated by machine learning in which the type of the footwear is added to the explanatory variable, the precision of the interpolated deficit data becomes high.

The classification unit 13 classifies the living-body information data on the basis of the attribute data stored in the storage unit 11. For example, the classification unit 13 classifies living-body information data of users having similar attributes into the same group on the basis of the attribute data.

For example, the classification unit 13 classifies a plurality of users on the basis of at least one of the attributes included in the attribute data. For example, the classification unit 13 classifies a plurality of users on the basis of any of attributes such as gender, height, and weight included in the attribute data. For example, the classification unit 13 may classify a plurality of users on the basis of any combination of attributes such as gender, height, and weight included in the attribute data. For example, the classification unit 13 may classify a plurality of users by machine learning using an algorithm such as a K-means method.

The aggregation unit 14 generates a table (also referred to as a living-body information table) obtained by aggregating the living-body information data related to each of the groups classified by the classification unit 13.

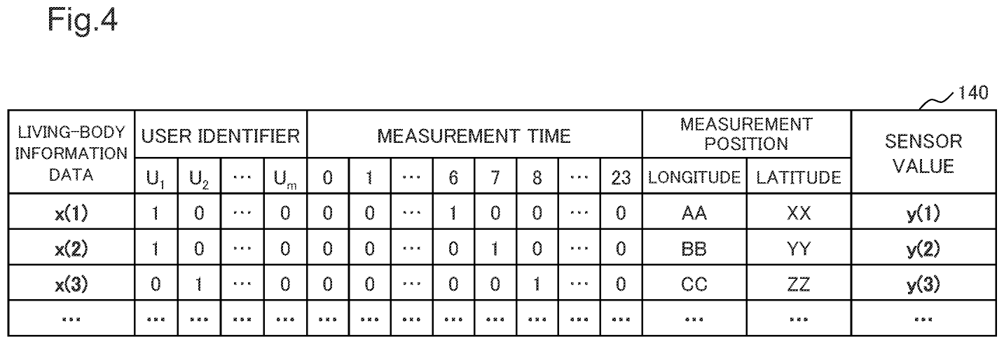

FIG. 4 is an example (living-body information table 140) of the living-body information table generated by the aggregation unit 14. The living-body information data included in the living-body information table 140 includes a user identifier, a measurement time, a measurement position, and a sensor value. For example, the living-body information data x(1) has a user identifier U₁, a measurement time of 6:00, a measurement position of (latitude AA, longitude BB), and a sensor value of y(1). Since the living-body information data is classified on the basis of the attribute at the stage of aggregation by the aggregation unit 14, the user identifier may not be included in the living-body information table.

The living-body information data is sparse data including many zeros. The measurement time of the sensor data is set for each user. In the present example embodiment, regarding living-body information data of a certain user, a sensor value at a measurement time at which sensor data is not measured is interpolated using a model generated by machine learning including a sensor value included in sensor data of another user classified into the same group. The deficit in the living-body information data does not need to be interpolated for all the measurement times, and may be interpolated for at least one measurement time.

The learning unit 15 generates a model for estimating interpolation data that interpolates the deficit in the living-body information data by using the correlation among the sensor value, the measurement time, and the measurement position included in the living-body information data of the user classified into the same group by the classification unit 13. For example, the learning unit 15 generates a model by machine learning with the measurement time and the measurement position as explanatory variables and the sensor value as an objective variable with respect to the living-body information data. For example, the learning unit 15 generates an estimation equation for estimating the interpolation data by using the correlation among the sensor value, the measurement time, and the measurement position included in the living-body information data of the user classified into the same group by the classification unit 13.

The learning unit 15 inputs the living-body information table aggregated in association with each of the plurality of groups from the aggregation unit 14. The learning unit 15 models the correlation among the sensor value, the measurement time, and the measurement position included in the living-body information data for each of the plurality of living-body information tables. For example, the learning unit 15 generates a model by machine learning with the measurement time and the measurement position as explanatory variables and the sensor value as an objective variable with respect to the living-body information data included in the living-body information table. For example, the learning unit 15 generates, for each of the plurality of living-body information tables, an estimation equation obtained by modeling the correlation among the sensor value, the measurement time, and the measurement position included in the living-body information data.

For example, the learning unit 15 generates a model for interpolating the living-body information data by machine learning in which the measurement time and the measurement position included in the living-body information data constituting the living-body information table are explanatory variables and the sensor value is an objective variable. For example, in a case where the measurement timing of the sensor value of a certain user is limited to the morning or the evening, it is possible to accurately grasp the living-body information of the user when there is the sensor data in the daytime. In the present example embodiment, the sensor value in the deficit time zone is interpolated by learning using the living-body information data of users having similar attributes. Even if the attributes are similar, it is assumed that the sensor values are different when the environment in which the sensor data is acquired is different. In the present example embodiment, it is possible to generate a model in consideration of the environment in which the sensor data is acquired by learning the living-body information data of the users having similar attributes including the measurement position of the sensor value.

For example, the learning unit 15 generates a model by machine learning using a deep planning method. For example, the learning unit 15 generates a model for interpolating the living-body information data by optimizing a structure (parameter, connection relationship between nodes, and the like) of a neural network (NN) by machine learning. Examples of the NN include a convolutional neural network (CNN) and a recurrent neural network (RNN). However, the learning method used by the learning unit 15 is not particularly limited as long as it is a method capable of interpolating the deficit in the living-body information data.

For example, the learning unit 15 generates a model by machine learning using a method such as singular value decomposition (SVD), matrix factorization (MF), or factorization machines (FM). For example, the learning unit 15 generates an estimation equation obtained by modeling the correlation between the measurement time and the measurement position included in the living-body information data for each of the plurality of living-body information tables using the FM method. The learning unit 15 may construct an estimation equation obtained by modeling the correlation between the measurement time and the measurement position including the user identifier. When the user identifier is included, the sensor value included in the living-body information data of the same user is preferentially learned, so that the precision of the interpolation data is further improved.

The following Expression 1 is an expression indicating the sensor value y(x) in a case where the living-body information table is regarded as a matrix V (i, j, and n are integers indicating a relationship of 1≤i<j≤n).

$$y ( x ) = {w}_{0} + {\sum }_{i = 1}^{n} {w}_{i} {x}_{i} + {\sum }_{i = 1}^{n} {\sum }_{j = i + 1}^{n} 〈 {v}_{i} , {v}_{j} 〉 {x}_{i} {x}_{j} \qquad ( 1 )$$

x in Expression 1 is a vector including the measurement time and the measurement position. w₀ in the first term on the right side of Expression 1 is a global bias. wᵢ models the intensity of the i-th variable.

The third term on the right side of Expression 1 corresponds to an intersection term of elements of the living-body information data x. The angle brackets of the third term on the right side of Expression 1 represent the inner product of vᵢ and vⱼ. vᵢ represents a vector of the i-th row of the matrix V, and vⱼ represents a vector of the j-th row of the matrix V. The inner product of vᵢ and vⱼ is represented by the following Expression 2 (f and k are integers).

$$〈 {v}_{i ,} {v}_{j} 〉 = {\sum }_{f = 1}^{k} {v}_{i , f} \cdot {v}_{j , f} \qquad ( 2 )$$

The parameters obtained by the machine learning are w₀, a vector w, and a matrix V shown in the following Expression 3. The dimension n is a hyperparameter.

w₀∈R, w∈Rⁿ, V∈R^{n×k} (3)

R in Expression 3 represents a real number. n represents the number of rows of the vector w and the matrix V, and k represents the number of columns of the matrix V.

The interpolation unit 16 (also referred to as an estimation unit) estimates the deficit value of the living-body information data using the model generated by the learning unit 15. The interpolation unit 16 interpolates the living-body information data including the deficit by using the estimated deficit value. For example, the living-body information data in which the deficit is interpolated is stored in the storage unit 11.

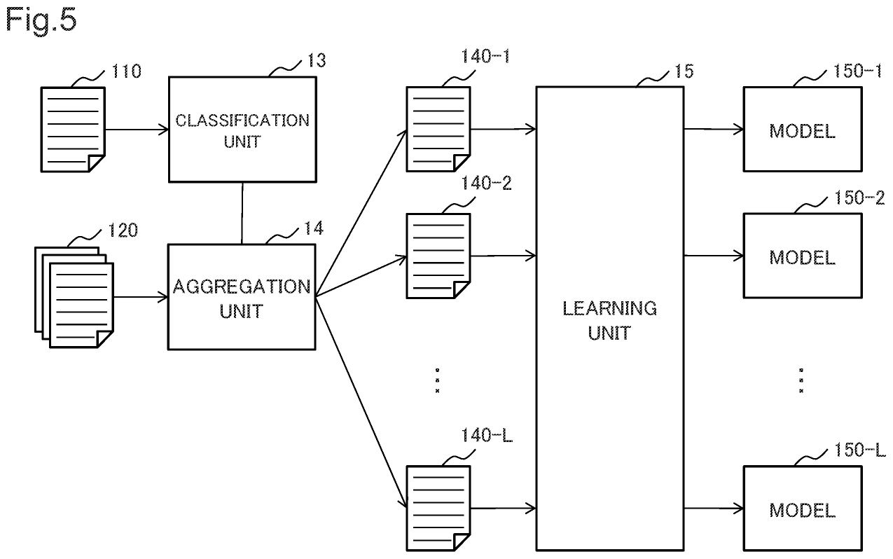

FIG. 5 is a conceptual diagram for explaining an example in which the data processing device 10 generates models (models 150-1 to 150-L) (L is a natural number). The classification unit 13 classifies users having similar attributes into the same group on the basis of at least one of the attributes included in the attribute table 110 stored in the storage unit 11. The aggregation unit 14 generates at least one of the living-body information tables 140-1 to 140-L by using living-body information data 120 included in each of the groups classified by the classification unit 13 (L is a natural number). The learning unit 15 generates each of the models 150-1 to 150-L using the correlation among the sensor value, 15 the measurement time, and the position included in the living-body information data for at least each one of the living-body information tables 140-1 to 140-L.

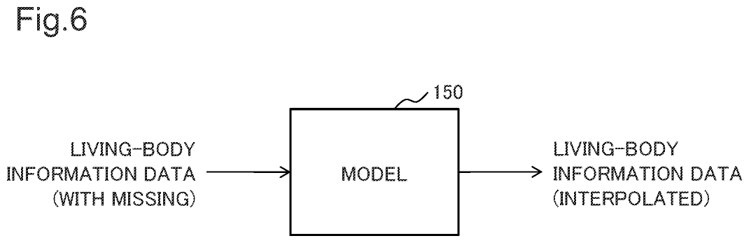

FIG. 6 is a conceptual diagram illustrating an example in which living-body information data having a deficit is input to the model 150 generated by the learning unit 15. When the living-body information data having a deficit is input to the model 150, the living-body information data in which the deficit is interpolated is output. For example, in a case where there is deficit in the daytime time zone in the living-body information data of a certain user, a sensor value at any time in the daytime time zone among the living-body information data classified into the same group is interpolated to the living-body information data of the user. As a result, in the living-body information data of the user, a deficit at at least any time is interpolated.

### Operation

Next, an example of the operation of the data processing device 10 of the present example embodiment will be described with reference to the drawings. The operation of the data processing device 10 includes a learning phase and an estimation phase. Hereinafter, each of the learning phase and the estimation phase will be individually described with the data processing device 10 as a subject of operation.

### [Learning Phase]

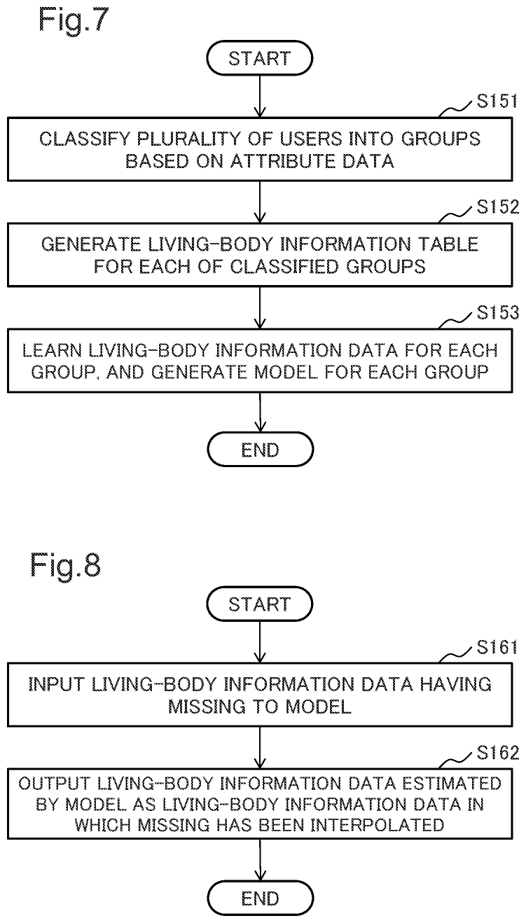

FIG. 7 is a flowchart for explaining an example of the learning phase. In FIG. 7, first, the data processing device 10 classifies a plurality of users into groups on the basis of attribute data (Step S151).

Next, the data processing device 10 aggregates the living-body information data of the users having similar attribute data for each of the classified groups to generate a living-body information table (Step S152).

Next, the data processing device 10 learns the living-body information data included in the living-body information table for each group, and generates a model for interpolating the living-body information data for each group (Step S 153).

### [Estimation Phase]

FIG. 8 is a flowchart for explaining an example of the estimation phase. In FIG. 8, first, the data processing device 10 inputs the living-body information data having a deficit to the model (Step S161).

Next, the data processing device 10 outputs the living-body information data estimated by the model as the living-body information data in which the deficit is interpolated (Step S 162).

Here, a modified example of the living-body information data will be described with an example. The following modified example is an example of improving the precision of aggregation and learning of the living-body information data by adding a further attribute to the living-body information data.

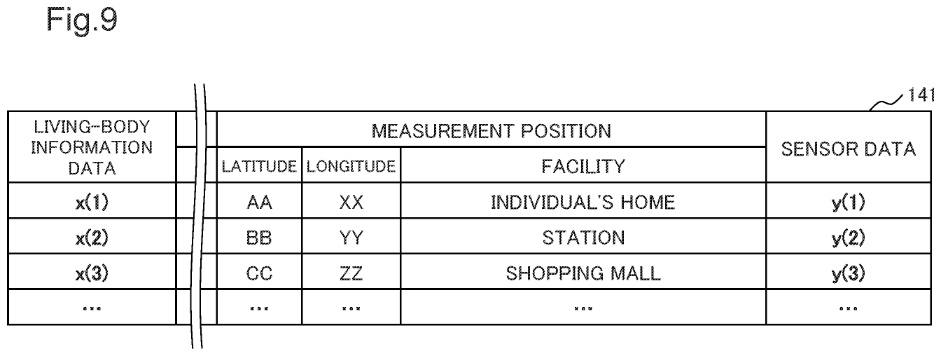

FIG. 9 illustrates an example in which a facility associated to a measurement position is added to the living-body information data on the basis of the latitude and longitude of the measurement position (living-body information table 141). For example, users having similar attributes and staying in the same facility are highly likely to measure similar sensor values. Therefore, if the living-body information data is classified on the basis of the facility associated to the measurement position or the model is generated by machine learning in which the facility is added to the explanatory variable, the precision of the interpolated deficit data becomes high.

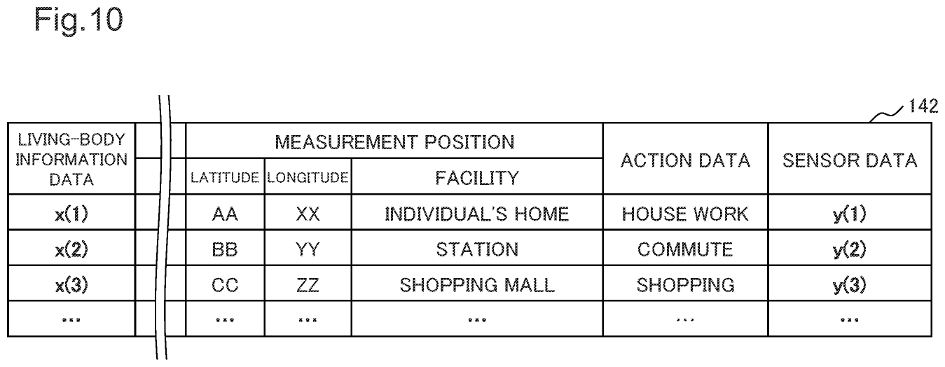

FIG. 10 illustrates an example in which an action that can be taken in the facility is added to the living-body information data in addition to the facility associated to the measurement position (living-body information table 142). For example, a user in an individual's home has a high probability of doing housework. The probability that the user at a station is on the way to work is high, and the probability that the user at a shopping mall is shopping is high. Therefore, if the living-body information data is classified on the basis of the action that can be taken in the facility or the model is generated by machine learning in which the action that can be taken in the facility is added to the explanatory variable, the precision of the interpolated deficit data becomes higher.

As described above, the data processing device according to the present example embodiment includes the storage unit, the classification unit, the aggregation unit, the learning unit, and the interpolation unit. The storage unit stores attribute data of each of the plurality of users and living-body information data of the plurality of users. The classification unit classifies the living-body information data on the basis on the attribute data stored in the storage unit. The aggregation unit generates a living-body information table obtained by aggregating living-body information data associated to each of the groups classified by the classification unit. The learning unit generates a model for estimating interpolation data that interpolates a deficit in the living-body information data by using a correlation among the sensor value, the measurement time, and the measurement position included in the living-body information data of the user classified into the same group by the classification unit. The learning unit models the correlation among the sensor value, the measurement time, and the measurement position included in the living-body information data for each of the plurality of living-body information tables. The interpolation unit (also referred to as an estimation unit) estimates a deficit value of the living-body information data using the model generated by the learning unit. The interpolation unit interpolates the living-body information data including the deficit by using the estimated deficit value.

According to the present example embodiment, by interpolating sensor values measured for at least one user with each other, it is possible to generate a model capable of interpolating the deficit in data relating to a living body with high precision.

In the present example embodiment, a model is generated using a correlation between a sensor value relating to a living body of a user and a measurement time and a measurement position of the sensor value on the basis of attributes of a plurality of users. For example, sensor values of users having the same attribute may be greatly different as long as the sensor values are measured at different positions even if the measurement times are the same. For example, the sensor value may show a different tendency depending on the measured scene, such as in a workplace, on a commuting route, on a way to lunch, or on a way to a meal after finishing work. In the present example embodiment, since a model associated to a scene in which a sensor value is measured can be generated, it is possible to interpolate a deficit that can be included in living-body information data with high precision.

### Second Example Embodiment

Next, a system according to a second example embodiment will be described with reference to the drawings. The system in the present example embodiment includes a wearable device, a terminal device, and a data processing device. Hereinafter, a gait measuring device mounted on an insole installed in footwear will be described as an example. The wearable device of the present example embodiment is not limited to the gait measuring device as long as the wearable device can measure data relating to the living body of the user.

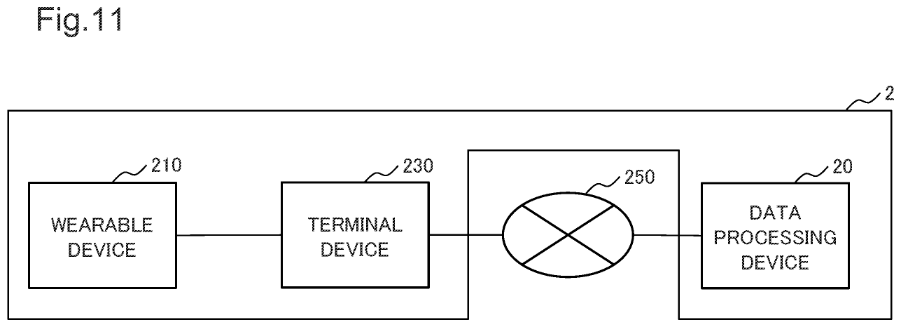

FIG. 11 is a block diagram for explaining an example of a configuration of a system 2 of the present example embodiment. The system 2 in the present example embodiment includes a wearable device 210, a terminal device 230, and a data processing device 20. The terminal device 230 is connected to the data processing device 20 via a network 250 such as the Internet or an intranet. For example, when the network 250 is an intranet, the network 250 may be added to the system 2 of the present example embodiment.

The wearable device 210 includes at least one sensor for measuring a sensor value included in the living-body information data. The wearable device 210 is worn by a user. The wearable device 210 measures a sensor value related to living-body information of the user wearing the wearable device 210.

The wearable device 210 transmits sensor data including the measured sensor value to the terminal device 230. For example, the wearable device 210 transmits sensor data to the terminal device 230 via a wireless communication function (not illustrated) conforming to a standard such as Bluetooth (registered trademark) or WiFi (registered trademark). For example, the wearable device 210 may transmit the sensor data to the terminal device 230 via a wire such as a communication cable. A method for transmitting the sensor data from the wearable device 210 to the terminal device 230 is not particularly limited.

For example, the wearable device 210 is achieved by a gait measuring device mounted on an insole installed on footwear. For example, the gait measuring device measures angular velocity and acceleration in three axial directions, and generates sensor data such as a stride length, a walking speed, a foot raising height, a grounding angle, a kicking angle, a foot angle, an inversion, and an eversion of the user.

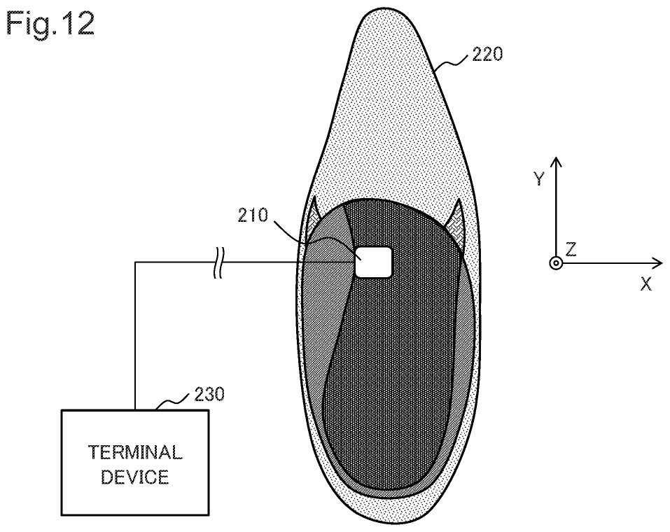

FIG. 12 is a conceptual diagram illustrating an example in which the wearable device 210 for measuring the gait is installed in a shoe 220. For example, the wearable device 210 is installed in an insole inserted into the shoe 220, and is disposed at a position associated to the back side of the arch of the foot. The position where the wearable device 210 is disposed may be a position other than the back side of the arch of the foot as long as the position is inside or on the surface of the shoe. The wearable device 210 may be installed on footwear, socks, or the like other than the shoe 220 as long as the gait can be measured.

The wearable device 210 is connected to the terminal device 230. The wearable device 210 includes at least an acceleration sensor and an angular velocity sensor. The wearable device 210 converts sensor values acquired by the acceleration sensor and the angular velocity sensor into digital data, and generates sensor data by giving a measurement time to the converted digital data. The sensor data may include a user identifier. The wearable device 210 transmits the generated sensor data to the terminal device 230.

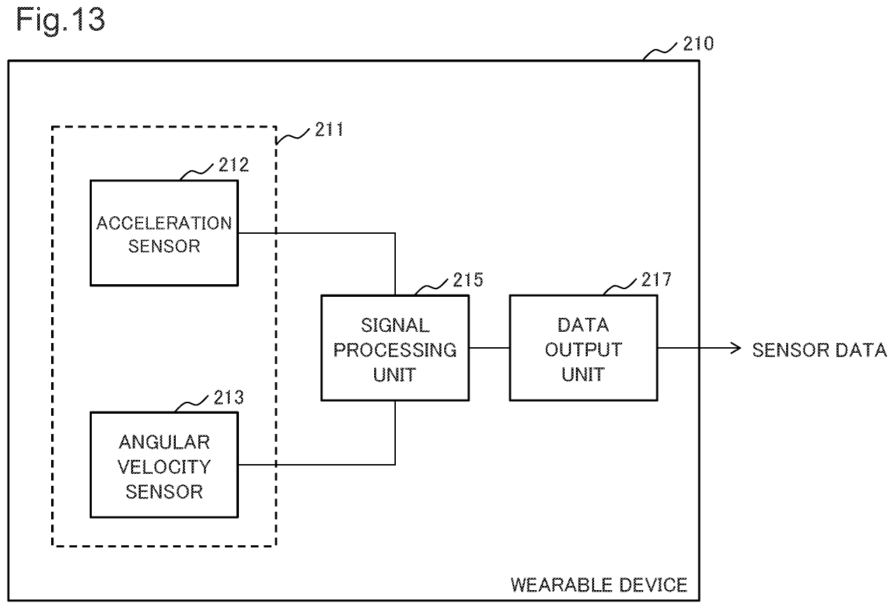

FIG. 13 is a block diagram illustrating an example of a configuration of the wearable device 210. The wearable device 210 includes an acceleration sensor 212, an angular velocity sensor 213, a signal processing unit 215, and a data output unit 217. The acceleration sensor 212 and the angular velocity sensor 213 constitute a sensor 211. For example, the sensor 211 is achieved by an inertial measurement unit (IMU).

The acceleration sensor 212 is a sensor that measures acceleration in three axial directions. The acceleration sensor 212 outputs the measured acceleration to the signal processing unit 215.

The angular velocity sensor 213 is a sensor that measures an angular velocity. The angular velocity sensor 213 outputs the measured angular velocity to the signal processing unit 215.

The signal processing unit 215 acquires raw data of each of the acceleration and the angular velocity from each of the acceleration sensor 212 and the angular velocity sensor 213. The signal processing unit 215 converts the acquired acceleration and angular velocity into digital data, and generates sensor data by giving a measurement time to the converted digital data. The measurement time is measured by a timer (not illustrated) or the like. In a case where the measurement time is given on the terminal device 230 side, the measurement time may not be included in the sensor data. The signal processing unit 215 may be configured to perform correction such as a mounting error, temperature correction, and linearity correction on raw data of the measured acceleration and angular velocity, and output a corrected sensor value. The signal processing unit 215 may give a user identifier to the sensor data. The signal processing unit 215 outputs the sensor data to the data output unit 217.

The data output unit 217 acquires sensor data from the signal processing unit 215. The data output unit 217 transmits the acquired sensor data to the terminal device 230. The data output unit 217 may transmit the sensor data to the terminal device 230 via a wire such as a communication cable, or may transmit the sensor data to the terminal device 230 via wireless communication. For example, the data output unit 217 transmits the sensor data to the terminal device 230 via a wireless communication function (not illustrated) conforming to a standard such as Bluetooth (registered trademark) or WiFi (registered trademark).

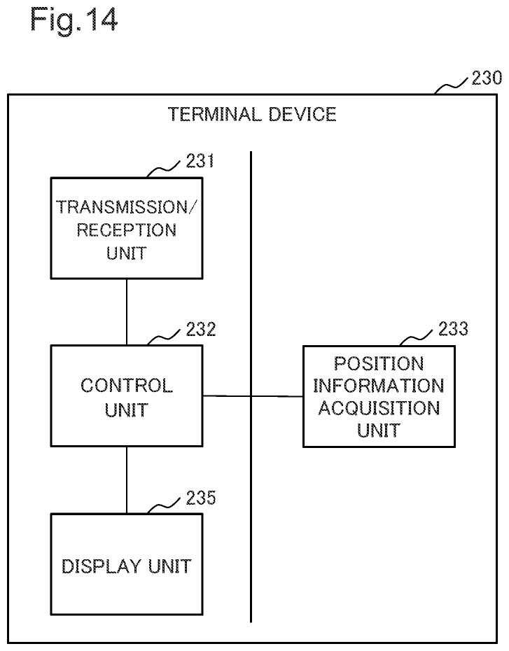

FIG. 14 is a block diagram illustrating an example of a configuration of the terminal device 230. The terminal device 230 includes a transmission/reception unit 231, a control unit 232, a position information acquisition unit 233, and a display unit 235.

The transmission/reception unit 231 receives sensor data from the wearable device 210. The transmission/reception unit 231 outputs the received sensor data to the control unit 232. The transmission/reception unit 231 receives the living-body information data from the control unit 232. The transmission/reception unit 231 transmits the received living-body information data to the data processing device 20. The timing at which the living-body information data is transmitted from the transmission/reception unit 231 is not particularly limited.

For example, the transmission/reception unit 231 transmits a request for interpolating the deficit in the living-body information data to the data processing device 20 according to processing of an application (for example, an application for analyzing living-body information) installed in the terminal device 230 or an operation of the user. The transmission/reception unit 231 receives the living-body information data transmitted in response to the request. For example, the living-body information data transmitted from the data processing device 20 is used for analysis of the living-body information in the application.

The control unit 232 acquires sensor data from the transmission/reception unit 231. When acquiring the sensor data, the control unit 232 acquires position information from the position information acquisition unit 233, and generates living-body information data by giving the acquired position information (measurement position) to the sensor data. In a case where the sensor data does not include the measurement time, the time of the timing at which the terminal device 230 generates the living-body information data may be given to the living-body information data as the measurement time. In a case where the wearable device 210 does not give the user identifier to the sensor data, the control unit 232 gives the user identifier to the living-body information data.

The position information acquisition unit 233 acquires position information. For example, the position information acquisition unit 233 acquires position information by a position measurement function using a GPS. The position information acquisition unit 233 may correct the position information acquired using the position measurement function with a sensor value measured by an angular velocity sensor, an angular velocity sensor, or the like (not illustrated). The position information acquired by the position information acquisition unit 233 is used for generation of living-body information data in the control unit 232.

The display unit 235 displays a user interface that receives a user's operation and an image related to an application or the like installed in the terminal device 230. For example, the display unit 235 displays an image of a result of processing the living-body information data received from the data processing device 20 by the application. The user who has viewed the image displayed on the display unit 235 can view the processing result of the application based on the living-body information data in which the deficit is interpolated by the data processing device 20. An image that can be displayed on a screen of a general smartphone, tablet, mobile terminal, or the like is displayed on the display unit 235, not limited to the processing result of the user interface or the application based on the living-body information data.

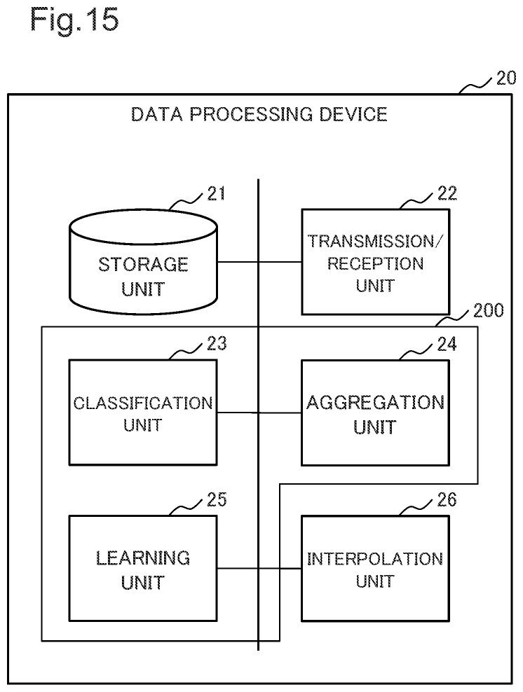

FIG. 15 is a block diagram illustrating an example of a configuration of the data processing device 20. The data processing device 20 includes a storage unit 21, a transmission/reception unit 22, a classification unit 23, an aggregation unit 24, a learning unit 25, and an interpolation unit 26. The classification unit 23, the aggregation unit 24, and the learning unit 25 constitute a model generation device 200. The storage unit 21, the classification unit 23, the aggregation unit 24, the learning unit 25, and the interpolation unit 26 are similar to the relevant configurations of the data processing device 10 of the first example embodiment, and thus detailed description thereof is omitted.

### Operation

Next, an operation of the system 2 of the present example embodiment will be described with reference to the drawings. The operation of the system 2 of the present example embodiment is roughly divided into an initial setting phase, a measurement phase, and a data processing phase. Hereinafter, each of the initial setting phase, the measurement phase, and the data processing phase will be individually described.

### [Initial Setting Phase]

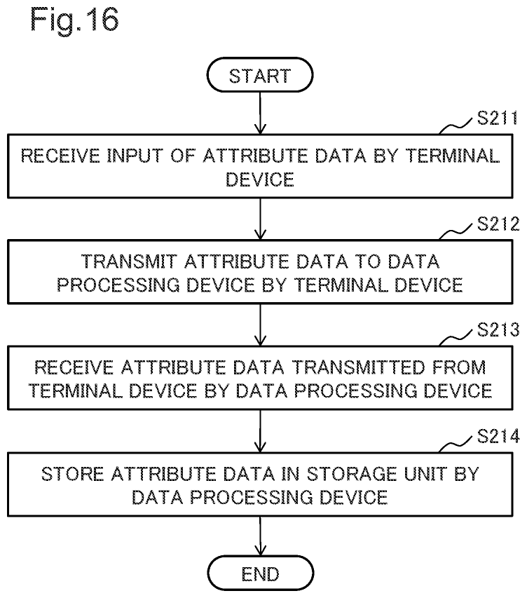

The initial setting phase is a phase in which the attribute data of the user is registered in the data processing device 20. FIG. 16 is a flowchart for explaining the initial setting phase.

In FIG. 16, first, the terminal device 230 receives the input of the attribute data of the user via the graphical user interface of the application displayed on the display unit 235 of the terminal device 230 (Step S211). For example, on the display unit 235 of the terminal device 230, a graphical user interface for accepting the attribute of the user is displayed, and the input of the attribute data by the user is accepted. For example, the terminal device 230 receives an input of attribute data such as the height, weight, and gender of the user. For example, the terminal device 230 may receive an input of attribute data such as a user's medical history.

Next, the terminal device 230 transmits the input attribute data to the data processing device 20 (Step S212).

Next, the data processing device 20 receives the attribute data transmitted from the terminal device 230 (Step S213).

Next, the data processing device 20 stores the received attribute data in the storage unit 21 (Step S214). The attribute data stored in the storage unit 21 is used for classification of the user.

### [Measurement Phase]

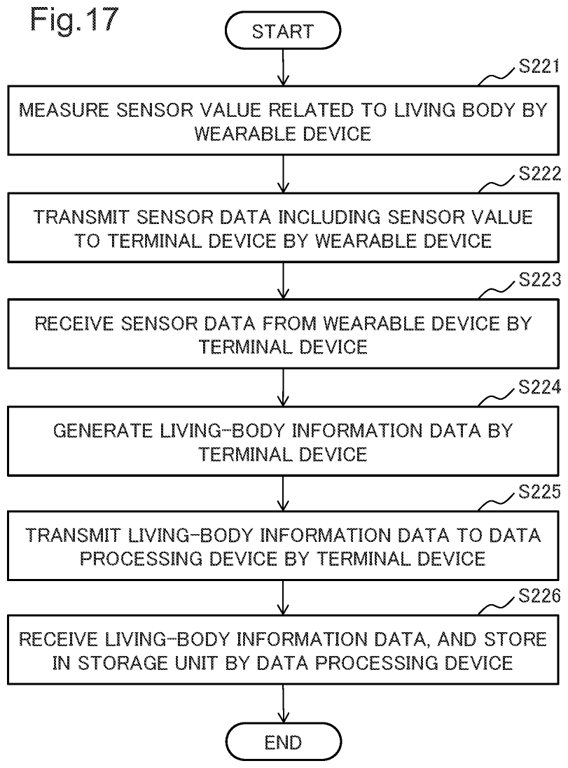

The measurement phase is a phase in which living-body information data based on sensor data measured by the wearable device 210 is stored in the data processing device. FIG. 17 is a flowchart for explaining a measurement phase.

In FIG. 17, first, the wearable device 210 measures a sensor value relating to a living body of a user wearing the wearable device 210 (Step S221).

Next, the wearable device 210 generates sensor data including the measured sensor value, and transmits the generated sensor data to the terminal device 230 (Step S222). For example, the wearable device 210 transmits sensor data in which a sensor value and a measurement time are associated with each other to the terminal device 230.

Next, the terminal device 230 receives the sensor data from the wearable device 210 (Step S223).

Next, the terminal device 230 acquires position information (measurement position) of the terminal device 230 in accordance with the reception of the sensor data, and generates living-body information data in which the user identifier, the sensor value, the measurement time, and the measurement position are associated (Step S224).

Next, the terminal device 230 transmits the generated living-body information data to the data processing device 20 (Step S225).

Next, the data processing device 20 receives the living-body information data transmitted from the terminal device 230, and stores the received living-body information data in the storage unit 21 (Step S226). The living-body information data stored in the storage unit 21 is used for generating a model for interpolating a deficit.

### [Data Processing Phase]

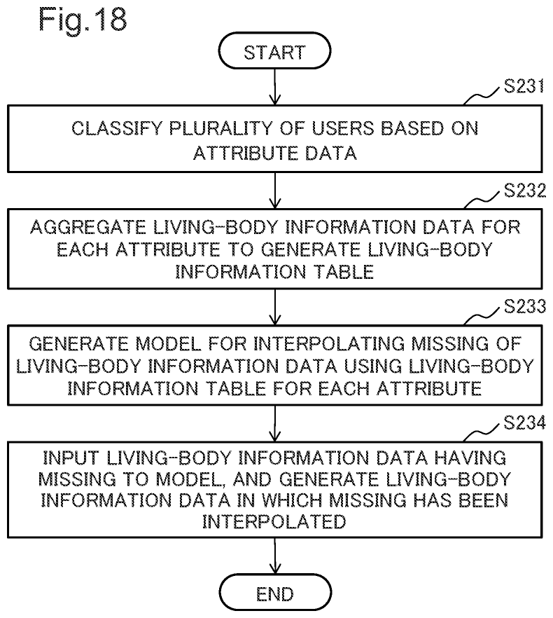

The data processing phase is a phase in which the deficit in the living-body information data is interpolated using the living-body information data of the plurality of users stored in the storage unit 21. FIG. 18 is a flowchart for explaining the data processing phase. In the description regarding the data processing phase of FIG. 18, components of the data processing device 20 will be described as an operation subject (in FIG. 18, the subject of the operation is omitted).

In FIG. 18, first, the classification unit 23 classifies a plurality of users on the basis of the attribute data stored in the storage unit 21 (Step S231). For example, the classification unit 23 clusters users having similar attributes into the same group.

Next, the aggregation unit 24 aggregates the living-body information data for each attribute on the basis of the classification by the classification unit 23 to generate a living-body information table (Step S232). For example, the aggregation unit 24 generates a living-body information table associated to each of the clustered groups.

Next, the learning unit 25 generates a model for interpolating the deficit in the living-body information data using the living-body information table for each attribute (Step S233). For example, the learning unit 25 generates, in the living-body information table for each attribute, an estimation equation that models a correlation among the sensor value, the measurement time, and the measurement position included in the living-body information data.

Next, the interpolation unit 26 (also referred to as an estimation unit) inputs living-body information data having a deficit to the model, and generates living-body information data in which the deficit is interpolated (Step S234). The living-body information data interpolated with the deficit is used in an application or the like using the living-body information data.

As described above, the system according to the present example embodiment includes a data processing device, a device (wearable device), and a terminal device. The device measures a sensor value. The terminal device generates living-body information data by giving a measurement time and a measurement position of the sensor value to the sensor value measured by the device.

According to the present example embodiment, it is possible to measure a sensor value relating to a living body of a user and generate living-body information data in which a measurement time and a measurement position are given to the measured sensor value.

### Third Example Embodiment

Next, a system according to a third example embodiment will be described with reference to the drawings. The system in the present example embodiment includes a plurality of wearable devices, a terminal device, and a data processing device. The system of the present example embodiment generates a model for estimating interpolation data which interpolates the deficit in living-body information data including sensor values measured by a plurality of wearable devices or the like.

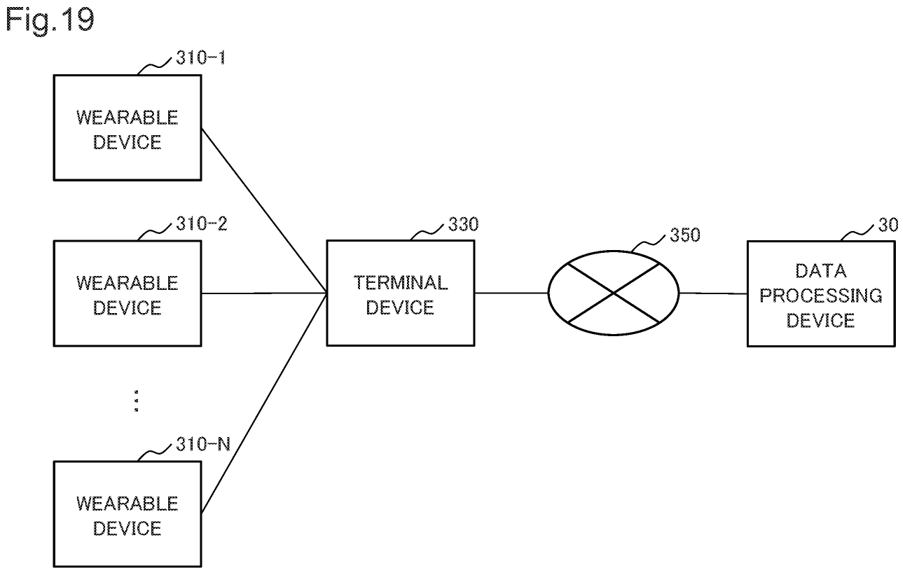

FIG. 19 is a block diagram for explaining an example of a configuration of a system of the present example embodiment. The system in the present example embodiment includes a plurality of wearable devices 310-1 to 310-N, a terminal device 230, and a data processing device 20 (N is a natural number). The terminal device 330 is connected to a data processing device 30 via a network 350 such as the Internet or an intranet. For example, when the network 350 is an intranet, the network 350 may be added to the system of the present example embodiment. Hereinafter, each of the plurality of wearable devices 310-1 to 310-N will be referred to as a wearable device 310 when not distinguished from each other.

For example, the wearable device 310 is achieved by a wristband-type device (also referred to as an activity meter) worn on a wrist or the like of the user. For example, the wristband-type device measures sensor data such as an activity amount, a pulse wave, sweating, and a body temperature of the user. For example, the wearable device 310 is achieved by an electroencephalograph. For example, the electroencephalograph measures sensor data such as brain waves, emotions, and stress of the user. For example, the wearable device 310 is achieved by a suit-type motion sensor (also referred to as a motion sensor). For example, the suit-type motion sensor measures sensor data such as a motion, a motion function, and a rehabilitation recovery degree of the user. The wearable device 310 listed here is an example, and does not limit the wearable device included in the system of the present example embodiment.

The wearable device 310 transmits sensor data including the measured sensor value to a terminal device 330. For example, the wearable device 310 transmits sensor data to the terminal device 330 via a wireless communication function (not illustrated) conforming to a standard such as Bluetooth (registered trademark) or WiFi (registered trademark). For example, the wearable device 310 may transmit the sensor data to the terminal device 330 via a wire such as a communication cable. A method for transmitting the sensor data from the wearable device 310 to the terminal device 330 is not particularly limited.

The terminal device 330 has a configuration similar to that of the terminal device 230 of the second example embodiment. The terminal device 330 receives sensor data from the wearable device 310. When receiving the sensor data, the terminal device 330 acquires the position information. For example, the terminal device 330 acquires position information by a position measurement function using a GPS. The terminal device 330 generates living-body information data by giving position information (measurement position) to the sensor data. The living-body information data includes an identifier of the wearable device 310 from which the sensor value is measured. The terminal device 330 transmits the generated living-body information data to the data processing device 30. The timing at which the living-body information data is transmitted from the terminal device 330 is not particularly limited.

The data processing device 30 has the same configuration as the data processing device 20 of the second example embodiment. The data processing device 30 receives the living-body information data from the terminal device 330. The data processing device 30 stores the received living-body information data. When receiving the request for the living-body information data from the terminal device 230, the data processing device 30 transmits the living-body information data associated to the request to the terminal device 330.

The data processing device 30 classifies the living-body information data on the basis of the stored attribute data. The data processing device 30 generates a table (also referred to as a living-body information table) obtained by aggregating the living-body information data associated to each of the classified groups. The living-body information data constituting the living-body information table includes sensor values measured by the plurality of wearable devices 310-1 to 310-N.

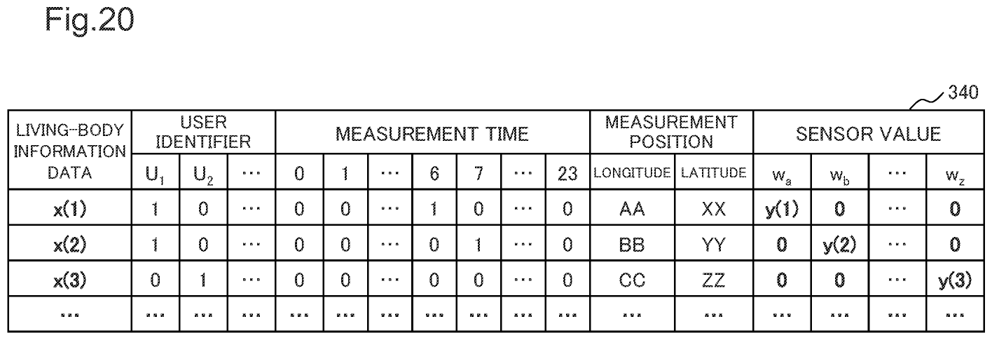

FIG. 20 illustrates an example (living-body information table 340) of the living-body information table generated by the data processing device 30. The living-body information data included in the living-body information table 340 includes a user identifier, a measurement time, a measurement position, and a sensor value. The sensor value includes values measured by the plurality of wearable devices 310-1 to 310-N.

The data processing device 30 models the correlation among the sensor value included in the living-body information data, the measurement time, and the measurement position with respect to the living-body information table aggregated in association with each of the plurality of groups. For example, the data processing device 30 generates a model by machine learning with the measurement time and the measurement position as explanatory variables and the sensor values measured by the plurality of wearable devices 310-1 to 310-N as objective variables with respect to the living-body information data included in the living-body information table. For example, the data processing device 30 performs weighting according to the distance with respect to the sensor values measured by the plurality of wearable devices 310-1 to 310-N. For example, the data processing device 30 increases the weight of the sensor values measured by the plurality of wearable devices 310-1 to 310-N as the distance is shorter.

The data processing device 30 estimates the deficit value of the living-body information data using the generated model. The data processing device 30 interpolates the living-body information data including the deficit by using the estimated deficit value.

For example, it is assumed that a measurement timing of sensor data by the wearable device 310 of a certain user is morning or evening, and a measurement timing of sensor data by the wearable device 310 of another user is daytime. In such a case, by interpolating sensor values measured by the wearable devices 310, it is possible to enrich data relating to living bodies of the users.

For example, it is assumed that a measurement timing of sensor data by a certain wearable device 310 of a certain user is morning or evening, and a measurement timing of sensor data by another wearable device 310 of the user is daytime. In such a case, by interpolating sensor values measured by the wearable devices 310, it is possible to enrich data relating to the user's living body.

As described above, the system of the present example embodiment includes a data processing device, at least one device (wearable device), and a terminal device. The at least one device measures a sensor value. The terminal device generates living-body information data by giving a measurement time and a measurement position of the sensor value to the sensor value measured by the device.

According to the present example embodiment, in addition to the measurement time and the measurement position, by modeling the correlation with the sensor values measured by the plurality of wearable devices, the precision of the data interpolated in the living-body information data can be improved.

A general wearable device is limited in performance such as a power supply and measurement precision because of being worn on a human body on a daily basis. Therefore, there is a limit to the precision of the sensor value measured by a single sensor. In the present example embodiment, a living-body data platform (also referred to as a multi-modal living-body sensor platform) combining a plurality of sensors can be constructed. According to the multi-modal living-body sensor platform, even if performance of individual sensors is not high, sensor values measured by the sensors can be combined to obtain highly accurate data.

### Fourth Example Embodiment

Next, a data processing device according to a fourth example embodiment will be described with reference to the drawings. The data processing device of the present example embodiment is different from the first to third example embodiments in that a model for estimating an assessment value or the like (also referred to as an evaluation value) of an exercise function index, a health index, or the like of a user is generated instead of a model for interpolating a deficit. The evaluation value is an index relating to a living-body characteristic of the user.

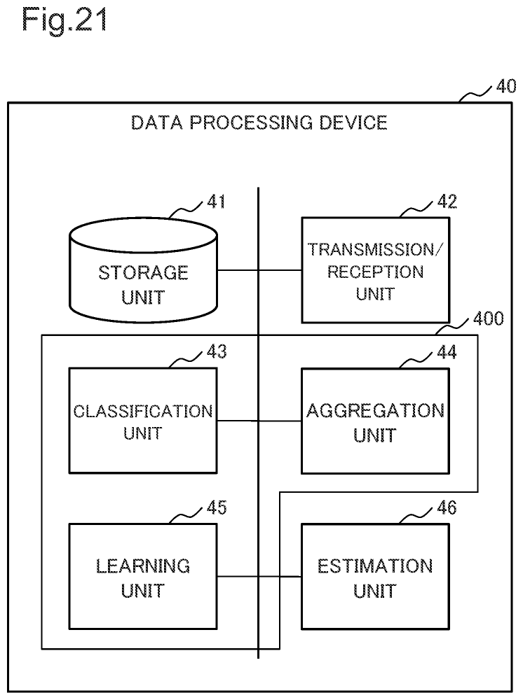

FIG. 21 is a block diagram illustrating an example of a configuration of a data processing device 40 according to the present example embodiment. The data processing device 40 includes a storage unit 41, a transmission/reception unit 42, a classification unit 43, an aggregation unit 44, a learning unit 45, and an estimation unit 46. The classification unit 43, the aggregation unit 44, and the learning unit 45 constitute a model generation device 400. The storage unit 41, the transmission/reception unit 42, the classification unit 43, and the aggregation unit 44 are similar to the relevant configurations of the data processing device 10 of the first example embodiment or the data processing device 20 of the second example embodiment, and thus, detailed description thereof is omitted.

The learning unit 45 generates a model for estimating a specific evaluation value using the sensor value included in the living-body information data classified into the same group by the classification unit 43 and the correlation between the measurement time and the measurement position. For example, the evaluation value is living-body information data in which a deficit is interpolated, or an assessment value such as an exercise function index or a health index. The evaluation value may be a questionnaire based subjective value such as the user's mood, physical condition, emotion, or the like.

The estimation unit 46 estimates an evaluation value such as an assessment value of the user by using the model generated by the learning unit 45. The evaluation value estimated by the estimation unit 46 is transmitted from the transmission/reception unit 42 to a terminal device (not illustrated). For example, the evaluation value transmitted to the terminal device is used by an application installed in the terminal device.

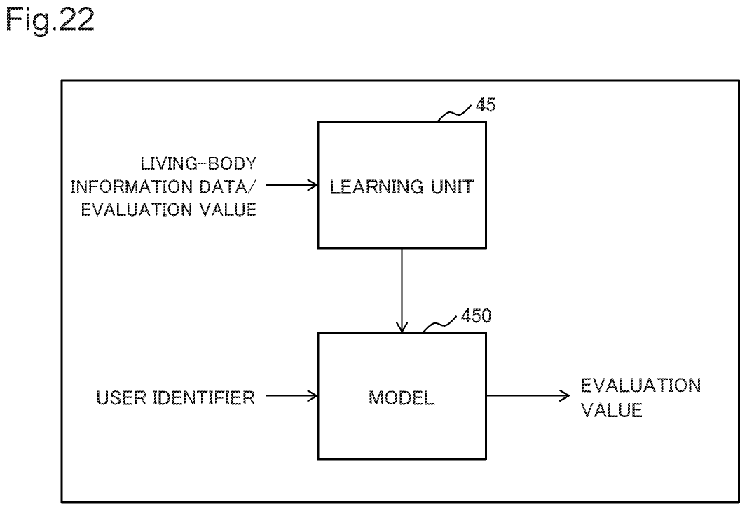

FIG. 22 illustrates an example in which a model 450 is generated by machine learning in which a plurality of pieces of living-body information data is used as explanatory variables and evaluation values such as assessment values and subjective values are used as objective variables, and a user identifier in the generated model 450 is input to estimate an evaluation value. For example, when a user identifier of a certain user is input to the model 450 generated using living-body information data of users having similar attributes, an evaluation value relating to the user is output from the model 450. For example, the output evaluation value may be transmitted to a terminal device (not illustrated) and processed by an application installed in the terminal device, thereby presenting content useful for the user to the user.

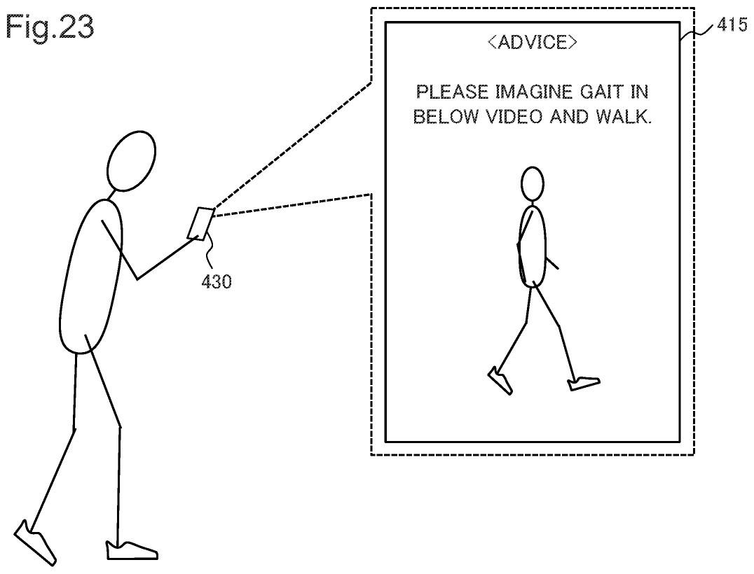

FIG. 23 is an example of displaying content based on the assessment value estimated by the model 450 on a screen 415 of a terminal device 430. In the example of FIG. 23, content including advice relating to the gait of the user is displayed on the screen 415 of the terminal device 430 on the basis of the assessment value estimated by the model generated by machine learning including the attribute of the footwear. The user who has viewed the content displayed on the screen 415 can obtain information leading to improvement of the exercise function index and the health index by performing exercise or the like according to the content.

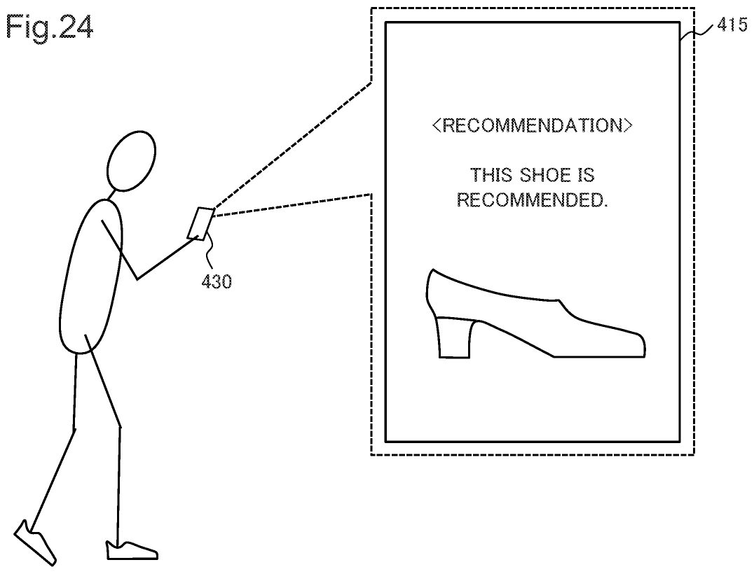

FIG. 24 is another example of displaying content based on the assessment value estimated by the model 450 on the screen 415 of the terminal device 430. In the example of FIG. 24, content including footwear recommended to the user is displayed on the screen 415 of the terminal device 430 on the basis of an assessment value estimated by a model generated by machine learning including an attribute of the footwear. The user who has viewed the content displayed on the screen 415 can obtain information such as footwear that matches his/her body by referring to the content.

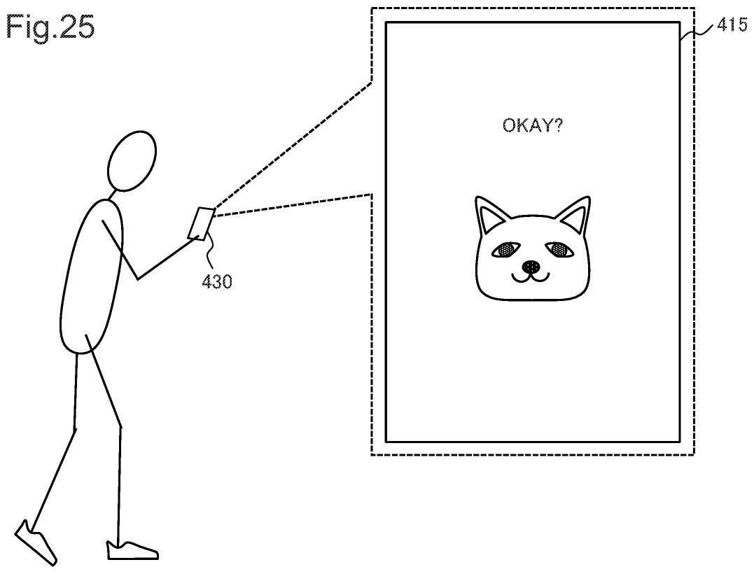

FIG. 25 is still another example of displaying content based on the subjective value estimated by the model 450 on the screen 415 of the terminal device 430. FIG. 25 is an example of displaying content matching the user's mood, physical condition, and emotion on the screen 415 of the terminal device 430 on the basis of an evaluation value estimated by the model. For example, in a case where a subjective value indicating that the user's emotion is unstable is output, the subjective value is displayed on the screen 415 of the terminal device 430 including an image of the user's favorite animal or character. When the user who has viewed the content displayed on the screen 415 refers to the content, there is a possibility that an unstable emotion is alleviated.

As described above, in the present example embodiment, the user identifier for identifying the user is added to the explanatory variable, and a model that estimates the evaluation value of the user is generated by machine learning using the evaluation value, which is the index relating to the living-body characteristic of the user, as the objective variable. According to the present example embodiment, it is possible to estimate an assessment value such as an exercise function index or a health index of a user, or an evaluation value such as a subjective value of the user, and display content associated to the evaluation value on a screen of a terminal device.

### Fifth Example Embodiment

Next, a model generation device according to a fifth example embodiment will be described with reference to the drawings. The model generation device of the present example embodiment has a simplified configuration of the model generation device 100 and the like included in the data processing device 10 of the first example embodiment. The data processing device can be configured only by the model generation device.

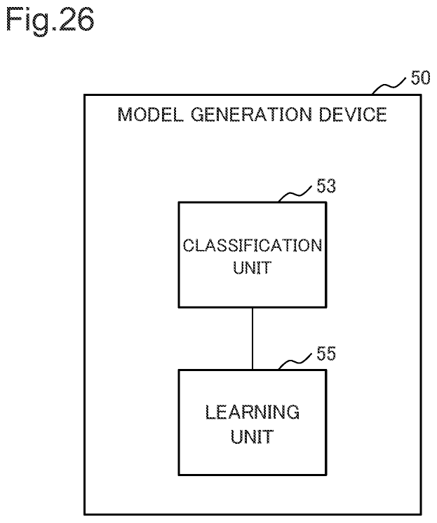

FIG. 26 is a block diagram illustrating an example of a configuration of a model generation device 50 of the present example embodiment. The model generation device 50 includes a classification unit 53 and a learning unit 55.

The classification unit 53 classifies at least one piece of living-body information data including a sensor value relating to a living body of the user and a measurement time and a measurement position of the sensor value into at least one group on the basis of an attribute of at least one user.

The learning unit 55 generates, for each group classified by the classification unit 53, a model for estimating living-body information data in which a deficit is interpolated by using a correlation among a sensor value included in the living-body information data, a measurement time, and a measurement position.

As described above, the model generation device (data processing device) of the present example embodiment includes the classification unit and the learning unit. The classification unit classifies at least one piece of living-body information data including a sensor value relating to a living body of the user and a measurement time and a measurement position of the sensor value into at least one group on the basis of an attribute of at least one user. The learning unit generates, for each group classified by the classification unit 53, a model for estimating living-body information data in which a deficit is interpolated by using a correlation among a sensor value included in the living-body information data, a measurement time, and a measurement position.

According to the present example embodiment, by interpolating sensor values measured for at least one user with each other, it is possible to generate a model capable of interpolating the deficit in data relating to a living body with high precision.

In one correspondence of the present example embodiment, the terminal device displays content including a processing result by the application using the living-body information data in which a deficit is stored by the data processing device on the screen. According to the present example embodiment, information useful for the user can be provided via content displayed on the screen of the terminal device.

### Sixth Example Embodiment

Next, an estimation device according to a sixth example embodiment will be described with reference to the drawings. The estimation device of the present example embodiment has a simplified configuration of the interpolation unit 16 and the like included in the data processing device 10 of the first example embodiment. The data processing device can be configured only by the estimation device.

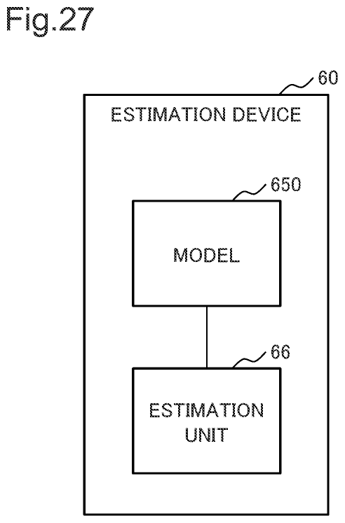

FIG. 27 is a block diagram illustrating an example of a configuration of an estimation device 60 according to the present example embodiment. The estimation device 60 includes a model 650 and an estimation unit 66.

The model 650 is a model generated by the data processing device of the first to fourth example embodiments or the model generation device of the fifth example embodiment. The model 650 is generated for estimating living-body information data in which a deficit is interpolated, for each group classified on the basis of the attribute of at least one user, by using a correlation among a sensor value included in the living-body information data, a measurement time, and a measurement position.

The estimation unit 66 inputs living-body information data having a deficit to the model 650, and estimates the living-body information data in which the deficit is interpolated.

As described above, the estimation device of the present example embodiment includes the model and the estimation unit. The model is generated for estimating living-body information data in which a deficit is interpolated, for each group classified on the basis of the attribute of at least one user, by using a correlation among a sensor value included in the living-body information data, a measurement time, and a measurement position. The estimation unit inputs living-body information data having a deficit to the model, and estimates the living-body information data in which the deficit is interpolated.

According to the present example embodiment, by interpolating sensor values measured for at least one user with each other, a deficit in data relating to a living body can be interpolated with high precision.

### Hardware

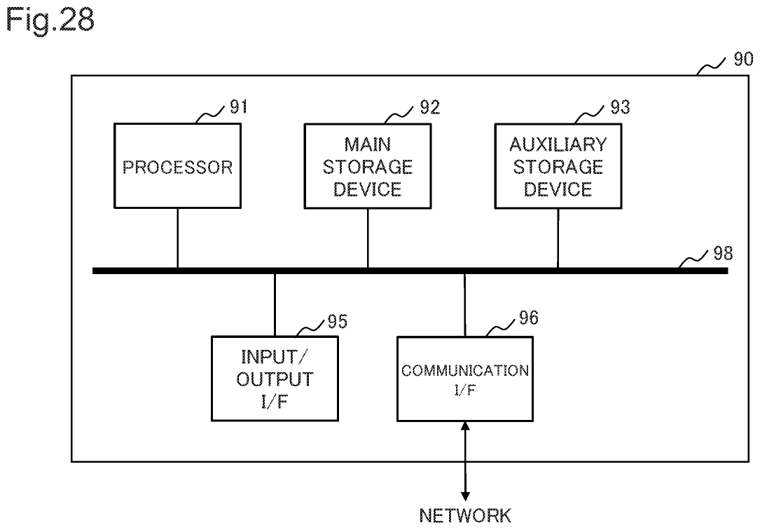

Here, a hardware configuration for executing processing of the data processing device according to each embodiment of the present invention will be described using an information processing device 90 of FIG. 28 as an example. The information processing device 90 in FIG. 28 is a configuration example for executing processing of the data processing device of each embodiment, and does not limit the scope of the present invention.

As illustrated in FIG. 28, the information processing device 90 includes a processor 91, a main storage device 92, an auxiliary storage device 93, an input/output interface 95, and a communication interface 96. In FIG. 28, the interface is abbreviated as an I/F. The processor 91, the main storage device 92, the auxiliary storage device 93, the input/output interface 95, and the communication interface 96 are data-communicably connected to each other via a bus 98. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like in the main storage device 92 and executes the developed program. In the present example embodiment, a software program installed in the information processing device 90 may be used. The processor 91 executes processing by the data processing device according to the present example embodiment.

The main storage device 92 has an area in which a program is developed. The main storage device 92 may be a volatile memory such as a dynamic random access memory (DRAM). A nonvolatile memory such as a magnetoresistive random access memory (MRAM) may be configured and added as the main storage device 92.

The auxiliary storage device 93 stores various data. The auxiliary storage device 93 includes a local disk such as a hard disk or a flash memory. Various data may be stored in the main storage device 92, and the auxiliary storage device 93 may be omitted.

The input/output interface 95 is an interface for connecting the information processing device 90 and a peripheral device. The communication interface 96 is an interface for connecting to an external system or device through a network such as the Internet or an intranet on the basis of a standard or a specification. The input/output interface 95 and the communication interface 96 may be shared as an interface connected to an external device.

An input device such as a keyboard, a mouse, or a touch panel may be connected to the information processing device 90 as necessary. These input devices are used to input information and settings. When the touch panel is used as an input device, the display screen of the display device may also serve as the interface of the input device. Data communication between the processor 91 and the input device may be mediated by the input/output interface 95.

The information processing device 90 may be provided with a display device for displaying information. In a case where a display device is provided, the information processing device 90 preferably includes a display control device (not illustrated) for controlling display of the display device. The display device may be connected to the information processing device 90 via the input/output interface 95.

The above is an example of the hardware configuration for enabling the data processing device according to each embodiment of the present invention. The hardware configuration of FIG. 28 is an example of a hardware configuration for executing arithmetic processing of the data processing device according to each embodiment, and does not limit the scope of the present invention. A program for causing a computer to execute processing regarding the data processing device according to each embodiment is also included in the scope of the present invention. Further, a recording medium in which the program according to each embodiment is recorded is also included in the scope of the present invention.

The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card, a magnetic recording medium such as a flexible disk, or another recording medium. When a program executed by the processor is recorded in a recording medium, the recording medium is associated to a program recording medium.

The components of the data processing device in each embodiment can be arbitrarily combined. The components of the data processing device of each example embodiment may be implemented by software or may be implemented by a circuit.

Although the present invention has been described with reference to the embodiments, the present invention is not limited to the above embodiments. Various modifications that can be understood by those skilled in the art can be made to the configuration and details of the present invention within the scope of the present invention.

### REFERENCE SIGNS LIST

10, 20, 30, 40 data processing device
11, 21, 41 storage unit
13, 23, 43, 53 classification unit
14, 24, 44 aggregation unit
15, 25, 45, 55 learning unit
16, 26 interpolation unit
22, 42 transmission/reception unit
46, 66 estimation unit
50, 100, 200, 400 model generation device
60 estimation device
210, 310-1 to N wearable device
211 sensor
212 acceleration sensor
213 angular velocity sensor
215 signal processing unit
217 data output unit
230, 330 terminal device
231 transmission/reception unit
232 control unit
233 position information acquisition unit
235 display unit

1. A data processing device comprising:
a memory storing instructions; and
a processor connected to the memory and configured to execute the instructions to:
classify living-body information data including a sensor value relating to a living-body of the user, a measurement time and a measurement position of the sensor value into a group similar to attributes of the user;
estimate a subjective value indicating biological characteristics of the user using a machine learning model that estimates the subjective value using the sensor value included in the living body information data classified into the same group and a correlation between the measurement time and the measurement position; and
display content matching the subjective value estimated by the machine learning model on a screen of a terminal device used by the user.

2. The data processing device according to claim 1, wherein
the processor is configured to execute the instructions to
estimate the subjective value using the machine learning model generated by machine learning using the subjective value collected on a questionnaire basis as objective variable, and
display the content matching the subjective value of the user on the screen of the terminal device based on the subjective value estimated by the machine learning model.

3. The data processing device according to claim 1, wherein
the subjective value is an indicator of a mood.

4. The data processing device according to claim 1, wherein
the subjective value is an indicator of a physical condition.

5. The data processing device according to claim 1, wherein
the subjective value is an indicator of an emotion.

6. The data processing device according to claim 1, wherein
the processor is configured to execute the instructions to
estimate the subjective value of the user using the machine learning model generated by machine learning in which a user identifier for identifying the user is included as an explanatory variable, and the subjective value indicating living-body characteristic of the user is an objective variable.

7. The data processing device according to claim 4, wherein
the processor is configured to execute the instructions to
display the content optimized for healthcare use according to the subject value to the terminal device used by the user.

8. A system comprising:
the data processing device according to claim 1; and
a wearable device configured to measure a sensor value relating to a living-body of a user; and
a terminal device configured to generate living-body information data in which a measurement time and a measurement position of the sensor value are added to the sensor value measured by the wearable device.

9. A data processing method executed by a computer, the method comprising:
classifying living-body information data including a sensor value relating to a living-body of the user, a measurement time and a measurement position of the sensor value into a group similar to attributes of the user;
estimating a subjective value indicating biological characteristics of the user using a machine learning model that estimates the subjective value using the sensor value included in the living body information data classified into the same group and a correlation between the measurement time and the measurement position; and
displaying content matching the subjective value estimated by the machine learning model on a screen of a terminal device used by the user.

10. A non-transitory program recording medium recorded with a program causing a computer to perform the following processes:
classifying living-body information data including a sensor value relating to a living-body of the user, a measurement time and a measurement position of the sensor value into a group similar to attributes of the user;
estimating a subjective value indicating biological characteristics of the user using a machine learning model that estimates the subjective value using the sensor value included in the living body information data classified into the same group and a correlation between the measurement time and the measurement position; and
displaying content matching the subjective value estimated by the machine learning model on a screen of a terminal device used by the user.
